(19) United States
(12) Patent Application Publication
(10) Pub. No.: US 2004/0038984 A1
(43) Pub. Date: Feb. 26, 2004

(54) Composition for male & female sexual arousal

(76) Inventors: Marie H. Harbeck, Carrollton, TX (US); Joseph F. Long, Austin, TX (US)

Correspondence Address: Marie H. Harbeck, 2805 Silverspring Road, Carrollton, TX, 75006, US

(21) Appl. No.: 10/225,012

(22) Filed: Aug. 22, 2002

(51) Int. Cl. A61K 31/519; A61K 31/4745

(52) U.S. Cl. 514/252.16; 514/283

(57) ABSTRACT

This inventon relates to compositions for stimulating sexual arousal in males and females and for male and female sexual arousal enhancement, and more particularly to the formaton of and treatment with said arousal, spermicidal and fungicidal composition to a male and female.

### BACKGROUND OF INVENTION

This invention relates to compositions for stimulating vaginal lubrication in females and sexual arousal in males and for male and female sexual arousal enhancement, and more particularly to the administration of and treatment of an arousal, spermicidal and fungicidal composition to a human male and stimulating vaginal lubrication and sexual arousal composition transdermally to a human female.

To the best of the applicant's knowledge the following is the most relevant prior art:

| | | | |
|---|---|---|---|
| U.S. Pat. No. | 5,945,117 | El-Rashidy, et al. | 1999 |
| U.S. Pat. No. | 6,031,002 | Wysor. et al. | 2000 |
| U.S. Pat. No. | 6,049,240 | See | 2000 |
| U.S. Pat. No. | 6,051,555 | Hadley | 2000 |
| U.S. Pat. No. | 6,193,992 | El-rashidy, et al. | 2001 |
| U.S. Pat. No. | 6,194,433 | Cutler | 2001 |

Studies have revealed that approximately 10 million women in the United States between the ages of 50 and 74 reported a lack of vaginal lubrication and sexual arousal on 229 million sexual intercourse occasions. In fact, it was found that over fifty-eight percent of 260 females surveyed were affected by sexual dysfunction and lack of vaginal lubrication, as defined as pain or discomfort during sexual intercourse, insufficient vaginal lubrication, delayed or non existent vaginal engorgement, increased time for arousal, diminished ability to reach orgasm, or diminished clitoral sensation.

Female sexual dysfunction increases with age, vascular illness, diabetes, thyroid dysfunction, onset of menopause, and hormonal imbalances and fluctuations.

Female sexual dysfunction has not been addressed sufficiently nor studied as extensively as male sexual dysfunction, due to the historical belief that female sexual dysfunction was related to lack of sexual libido.

There is a growing body of evidence that women with sexual dysfunction have common physiologic abnormalities, such as vasculogenic female sexual dysfunction, contributing to their overall sexual health problems. Present day management of women with sexual arousal disorder, especially those with diminished vaginal lubrication and lack of sexual arousal, as well as a lack of vaginal and clitoral engorgement may be impaired by their vasculogenic dysfunction resulting in inadequate blood flow to the clitoral glans, labia minorra and vulval areas.

Most recently, studies with females using the present invention have shown such females to have excellent vascular bioavailability. For example, it was found in one study that an extravaginal application of 10.8 mg of Yohimbe incorporated in a cream formula was optimum for producing a vasculogenic effect on vaginal and clitoral blood flow in a 36 year old female. A significant sexual readiness was evident in the female subject fifteen minutes after application, which produced clitorial erectogenesis, vaginal engorgement, vaginal lubrication, labia minorra engorgement, as well as very aroused sexual responses and readiness for sexual intercourse. During intercourse, the female subject reported that orgasm was faster to attain, whilst the male partner reported that he experienced a very prolonged penile erection due to the transdermal application on his penis with the said formulation. Both female and male subjects reported that sexual arousal was still high three hours after the initial application. Failure or loss of sexual desire to attain orgasm is encountered more frequently in women than in men. Yet, effective, safe, non toxic formulations for treating men and women who have difficulty in attaining orgasm have not been available to them.

The pharmaceutical industry is expanding its efforts in order to provide treatment for female and male sexual dysfunction and arousal disorders, thus methods of stimulating dopamine receptors in the mid-brain region of a human female are administered orally and intravenously. Toxic, nausea inducing synthetic vascular dilator compositions and methods employed continue to increase yearly.

El-rashidy, et al, U.S. Pat. No. 5,945,117, 1999, the disclosure of which has shown a method of ameliorating sexual dysfunction by administering to a female apomorphine, or beta.-cyclodextrin or acceptable acid addition salt as an oral dosage form and in a sufficient amount to increase blood flow to the vaginal wall in amounts less than the amount that induces substantial nausea. This method of stimulating dopamine receptors in the mid-brain region of a human female during sexual activity given orally and administered intravenously in doses in sufficient amount less than induces substantial nausea, has shown to have very poor bioavailability in providing a practical therapeutic use in the areas of female sexual dysfunction.

Wysor, et al, U.S. Pat. No. 6,031,002, 2000, discloses the use of prostaglandin, papaverine, sodium nitroprusside, phenoxybenzanine, phentolamine, prazosin, and vocative neuropathies and their natural and synthetic analogs, dimethylsulfoxide and its analogs, a vasodilator such as alprostadil, a prostaglandin of the E series and its analogs. Prostaglandins have been used to treat male impotence and in the treatment of females who exhibit nuerotic symptoms which may cause female sexual dysfunction. The method of topical use with minimum toxicity levels as disclosed in the above mentioned patent dilates the vaginal blood vessels within three to four minutes. It has shown that without sufficient time for vaginal lubrication to take place, sexual penetration will be painful for the female subject, even in an aroused state, based on psychologic, hormonal interventions. Antihypertensive drugs, tranquilizers and antidepressants, and many other agents may all cause decreased erectile capacity.

See, U.S. Pat. No. 6,046,240, 2000, discloses the use of prostanoid compounds, prostaglandin E-1, prostaglandin A, prostaglandin F, including PGF-2, PGF-D, prostacylins, thromboxanes, leukotrienes, 6-keto-PGE-1 derivatives, carbacyclin derivatives, PGD-2 derivatives and the like, and synthetic analogs of any portioned compounds, in a methylcellulose vehicle, has shown that the doses of portioned compounds should be maintained at relatively low doses due to the potential toxicity of the portioned compounds caused by rapid absorption of the portioned compounds across the vaginal tissue. Antihypertensive drugs, tranquilizers and antidepressants, and many other agents may all cause decreased erectile capacity.

Hadley, U.S. Pat. No. 6,051,555, 2000, discloses the use of peptides from a group consisting of, norleucine, aspartic acid, histidine, d-phenylalanine, arginine, tryptophan, lysine, glycine, proline, tyrosine, and serine. As shown in male human testing, each of the peptides induced an erection in the human male, which was enhanced in combination with a penile injection of 10 mg of peptide per ml of physiological saline. As shown, no human female testing has been done. It is shown that the peptides in this invention may be used in animal husbandry breeding programs.

El-rashidy, et al, U.S. Pat. No. 6,193,992, 2001, discloses apormorphine hydrochloride salt and beta.-cyclodextrin ( beta.-cyclodextrin is hydroxypropyl-beta-cyclodextrin) is administered in a sublingual dose. As shown in this disclosure, the use of dopamine receptors in the mid-brain region of the human female are used to stimulate the female during sexual activity.

Cutler, U.S. Pat. No. 6,194,433, 2001, discloses treating female sexual dysfunction with oral administrated flosequinan with flosequinan also administered cutaneously and transurethrally, and contemplates using a variety of quinoline derivatives such as methylthio, methylsulphinyl to smooth muscle cells to increase blood flow. It is shown that with oral administration of flosequinan, peak plasma concentrations of flosequinan are observed 1-2 hours following oral administration.

The problem with the use of the compositions of the cited prior art such as El-rashidy, U.S, Pat. No. 5,945,117, for treating female sexual dysfunction are their chemical structures which are known to cause to cause toxicity and substantial nausea, if not administered under strict supervisory conditions.

Unfortunately, in the compositions of the prior art the daily plasma concentrations of actives must be maintained via daily oral adminstration within the plasma of female subjects relating to the test subjects body weight.

The said cited prior art compositions, such as Cutler, U.S. Pat. No. 6,194,433, do not employ an organic, water-based emulsion as the vehicle for delivering organic vaginal engorgement additives to stimulate female and male sexual enhancement and arousal.

The said prior art compositions, See, U.S. Pat. No. 6,046,240, do not employ the use of an organic topical additive that is a non toxic female and male arousal vehicle.

The said prior art compositions, El-rashidy, et al, U.S. Pat. No. 5,945,117, Wysor, U.S. Pat. No. 6,031,002, See, U.S. Pat. No. 6,046,240, Hadley, U.S. Pat. No. 6,051,555, El-rashidy, et al, U.S. Pat. No. 6,193,992, do not employ a spermicidal additive in their female sexual enhancement and arousal compositions.

We found no prior art compositions that employed a spermicidal additive or a fungicidal additive in their female sexual enhancement and arousal compositions.

In terms of effectiveness, we found no prior art compositions that provide satisfactory skin penetration and supplying properties for female and male sexual arousal within six to ten minutes of applying said compositions.

We found no prior art that claimed a composition to be used as a condom lubricant to enhance male sexual function and arousal.

We found no prior art compositions to provide a spermicidal or fungicidal and male arousal additive in the form of a condom lubricant.

We found no prior art compositions to treat female sexual dysfunctions with a spermicical or fungicidal compound extravaginally.

We found no prior art compositions that claimed to have been tested extensively on human subjects as the present invention has.

El-rashidy, et al, U.S. Pat. No. 6,193,992, uses apomorphine which is a selective dopamine receptor agonist that has been widely utilized as an emetic agent, sedative, antiparkinsonian agent and a behaviour altering agent, but the effect of apormorphine on human female sexual functionality appears not to have not been previously investigated. El-rashidy, et al, U.S. Pat. No. 6,193,992, utilizes the administration of an anti emetic agent in conjuction with apormorphine oral administration to prevent nausea.

Wysor, et al, U.S. Pat. No. 6,031,002, employs prostoglandins in a crosslinked hydroxyl group which is insoluble in water based formulations.

We found no prior art compositions that claimed to be absorbed into the tissues within 39 seconds of application in male and female subjects.

We found no prior art compositions that claimed a near neutral composition as in our composition which has a pH of 6.9.

Some prior art compositions, See, U.S. Pat. No. 6,046,240, use egg yolk phospholipids and soybean phospholipids with their formulations which are known to cause allergic reactions in some human females and males.

We found no prior art compositions that use formulations whereby the sexual arousal state presents itself within the female within six minutes of topical application and within the male within fifteen minutes of topical application.

We found that compositions of this invention can be safely reapplied as often as needed for individual sexual response requirements.

Objectives and advantages of the present invention are:

a) to provide compositions for treating female and male sexual dysfunction without toxic formulations that are known to cause nausea,

b) to provide formulations whereby daily plasma concentrations of actives do not need to be maintained via daily oral administration,

c) to provide a water-based emulsion as the vehicle for delivering additives to stimulate female and male sexual enhancement and arousal,

d) to provide a topical additive that is a non-toxic female and male arousal vehicle,

e) to provide for use of a spermicidal additive in the female and male sexual enhancement and arousal composition,

f) to provide the use of a fungicidal vehicle within the female sexual enhancement and arousal formulation,

g) to provide for the use of both a fungicidal and spermicidal vehicle within the female sexual enhancement and arousal formulation,

h) to provide satisfactory skin penetration and supplying properties that provide female and male sexual arousal within six to fifteen minutes of applying said composition,

i) to provide a female sexual arousal additive, plus a spermicidal additive and a fungicidal additive that may be applied in an extravaginal formulation;

j) to provide an additive that can be used as a condom lubricant to enhance male sexual function and arousal.

k) to provide a spermicidal additive with a female arousal enhancement additive in the form of a transdermal application,

l) to provide a fungicidal additive with a male arousal enhancement additive in the form of an internal condom lubricant,

m) to provide a spermicidal, fungicidal and a female and male arousal additive in the form of an external condom lubricant,

n) to provide a female and male sexual enhancement and arousal formulation that has been tested on female and male subjects,

o) to provide female and male sexual enhancement and arousal compositions that are free of emetic agents, sedative, antiparkinsonian agents and a behaviour altering agents;

p) to provide female and male sexual enhancement and arousal compositions in a water based formulation for swift tissue penetration,

q) to provide female and male sexual enhancement and arousal formulations that are rapidly absorbed,

r) to provide female and male sexual enhancement and arousal formulations for safe extravaginal application,

s) to provide non tissue aggravating components within the female and male sexual enhancement and arousal formulations,

t) to provide formulations for female and male sexual enhancement and arousal whereby the sexual arousal state presents itself within the human female within a few minutes of topical application,

u) to provide organic formulations whereby the sexual arousal state of the female and male lasts for 2 to 3½ hours after topical administration,

v) to provide female and male sexual arousal formulations that can be safely reapplied as often as needed for individual sexual response requirements,

w) to provide a female and male sexual arousal formulation that can incorporate within its components fragrant plant oils for the users pleasure,

x) to provide a formulation for male sexual arousal that can be used to lubricate condoms.

y) to provide a formulation for female and male sexual arousal that can be used to lubricate condoms with a composition comprising a male sexual arousal component, and a spermicidal and fungicidal component.

Thus, an urgent need exists in the field of male and female sexual function and arousal enhancement for organic additives that will be readily accepted by the male and female body and be without undesirable side-effects, to enhance male and female sexual arousal, increase nerve stimulation and blood flow for enhanced clitoral erection, vaginal lubrication, as well as vaginal engorgement in a female and penile engorgement and erection in the male.

Further advantages of this invention are the employment of a neutral 6.9 pH water-based formulation that starts to penetrate the tissues within 39 seconds of application. Still further objects and advantages will become apparent from a consideration of the ensuing descriptions.

### SUMMARY OF THE INVENTION

In accordance with the present invention a male and female sexual arousal enhancement composition for stimulating vaginal lubrication, vaginal, clitoral engorgement and penile engorgement including the administration of spermicidal and fungicidal vehicles within the formulation for applying extravaginally, transdermally, and as a condom lubricant, comprising, water, cocoa butter, pro lipo H166.0, glycerol monostearate, carbomer, jojoba oil, and sodium behenoyl lactylate, and an aphrodisiac chosen from a group including Yohimbe and Viagra, and a spermicidal such as nonoxynol 9, and a fungicidal such as clotrimazole.

### DESCRIPTION OF THE INVENTION

It is an objective of the invention to provide compositions for treating male and female sexual dysfunction without toxic formulations that are known to cause nausea.

It is an objective of the invention to provide formulations whereby daily plasma concentrations of actives do not need to be maintained via daily oral administration.

It is an objective of the invention to provide a water-based emulsion as the vehicle for delivering additives to stimulate male and female sexual enhancement and arousal.

It is an objective of the invention to provide a topical additive that is a non-toxic male and female arousal vehicle.

It is an objective of the invention to provide for use of a spermicidal additive in a male and female sexual enhancement and arousal composition.

It is an objective of the invention to provide the use of a fungicidal vehicle-within a male and female sexual enhancement and arousal formulation.

It is an objective of the invention to provide for a use of both a fungicidal and spermicidal vehicle within a male and female sexual enhancement and arousal formulation.

It is an objective of the invention to provide satisfactory skin penetration and supplying properties that provide male and female sexual arousal within six to fifteen minutes of applying said composition.

It is an objective of the invention to provide a male sexual arousal additive, plus a spermicidal additive and a fungicidal additive that may be applied in an extravaginal and penile formulation.

It is an objective of the invention to provide an additive that can be used as a condom lubricant to enhance male sexual dysfunction and arousal enhancement.

It is an objective of the invention to provide a spermicidal additive with a female arousal enhancement additive in the form of a condom lubricant.

It is an objective of the invention to provide an additive that can be used as a condom lubricant to enhance male sexual arousal.

It is an objective of the invention to provide a spermicidal, fungicidal and female arousal additive in the form of a transdermal application.

It is an objective of the invention to provide a male and female sexual enhancement and arousal formulation that has been tested on male and female subjects.

It is an objective of the invention to provide male and female sexual enhancement and arousal compositions that are free of emetic agents, sedative, antiparkinsonian agents and behaviour altering agents.

It is an objective of the invention to provide male and female sexual enhancement and arousal compositions in a water based, formulation for swift tissue penetration.

It is an objective of the invention to provide female sexual enhancement and arousal formulations that are rapidly absorbed.

It is an objective of the invention to provide male and female sexual enhancement and arousal formulations for extravaginal application.

It is an objective of the invention to provide non tissue aggravating components within the male and female sexual enhancement and arousal formulations.

It is an objective of the invention to provide formulations for male and female sexual enhancement and arousal formulations whereby the sexual arousal state presents itself within the male and female within a few minutes of topical application.

It is an objective of the invention to provide organic formulations whereby the sexual arousal state of the male and female lasts for 2 to 3½ hours after topical administration.

It is an objective of the invention to provide male and female sexual arousal formulations that can be safely reapplied as often as needed for individual sexual response requirements.

It is an objective of the invention to provide a male and female sexual arousal formulation that can incorporate within its components fragrant plant oils for the users pleasure.

It is an objective of the invention to provide a formulation for male sexual arousal that can be used to lubricate condoms.

It is an objective of the invention to provide a formulation for male and female sexual arousal that can be used to lubricate condoms with a composition comprising a male and female sexual arousal component, and a spermicidal and fungicidal component.

The invention starts with a water miscible cream base. The water miscible cream base that comprises approximately:

77.0 grams water,

1.0 grams cocoa butter,

2.0 grams pro lip H166.01 purchased from Lucas Meyer BeautyEssentials, E. A. E. LaTuilleri, F77500 Chelles, France,

2.0 grams glycerol monostearate,

2.0 grams acritamer carbomer purchased from R.I.T.A Corporation, 1725 Kilkenny Conn., Woodstock, Ill. 60098,

1.0 gram jojoba oil, and

2.0 grams sodium behenoyl lactylate, compounded as follows:

place water and acritamer carbomer in a partially submerged container over hot water to form a first mixture, vigorously stirring said mixture over hot water until dissolved;

simultaneously placing cocoa butter, pro lipo H166.01, glycerol monostearate, jojoba oil and sodium behenoyl lactylate, in another partially submerged container over hot water to form a second mixture;

stirring said second mixture over said hot water until dissolved, removing said mixtures from heat and blending both said mixtures with stirring until said second mixture cools, whereby the ingredients are formed into a cream base.

All the following different compositions start with the same cream base and many similar compositions or formulations will be made as other aphrodisiacs evolve.

Transdermal cream number 1 comprises:

2000 parts of cream base mixed with 50 parts Viagara TM.

Transdernal cream number 2 comprises:

2000 parts cream base mixed with 100 parts Viagara TM.

Transdermal cream number 3 comprises:

2000 parts cream base mixed with 140 parts yohimbe.

Transdermal cream number 4 comprises:

2000 parts cream base mixed with 280 parts yohimbe.

Transdermal cream number 5 comprises:

5000 parts cream base mixed with 280 parts yohimbe,and 100 parts nonoxynol 9.

Transdermal cream number 6 comprises:

5000 parts cream base mixed with 280 parts yohimbe and 50 parts clotrimazole.

Transdermal cream number 7 comprises:

5000 parts cream base mixed with 280 parts yohimbe and 100 parts nonoxynol 9 and 50 parts clotrimazole.

Transdermal cream number 8 comprises:

2000 parts cream base mixed with 25 parts Viagara Tm.

Transdermal cream number 9 comprises:

5000 parts cream base mixed with 100 parts Viagara TM, and 50 parts clotrimazole,

Transdermal cream number 10 comprises:

5000 parts of cream base mixed with 100 parts Viagara TM, and 100 parts nonoxynol 9.

Transdermal cream number 11 comprises:

5000 parts of cream base mixed with 50 parts Viagara TM, 100 parts nonoxynol 9, and 50 parts clotrimazole.

Transdernal cream number 12 comprises:

5000 parts of cream base mixed with 25 parts Viagara TM.

Transdermal cream number 13 comprises:

5000 parts of cream base mixed with 100 parts of Viagara TM, and 148 parts yohimbe.

Transdermal cream number 14 comprises:

2000 parts of cream base mixed with 50 parts of Viagara Tm, and 280 parts yohimbe.

Transdermal cream number 15 comprises:

5000 parts of cream base mixed with 50 parts of Viagara TM, 200 parts of yohimbe, and 100 parts nonoxynol 9.

Transdermal cream number 16 comprises:

5000 parts cream base mixed with 50 parts of Viagara TM, 10.8 parts yohimbe, and 50 parts clotrimazole.

Transdermal cream number 17 comprises:

5000 parts of cream base mixed with 100 parts Viagara Tm, 280 parts yohimbe, 50 parts clotrimazole and 200 parts nonoxynol 9.

All the above compositions may be incorporated in said cream base by finely grinding any solid additives and mixing by stirring in a well known manner.

### Conclusion, Ramification, and Scope

Although the description above contains many specificities, these should not be construed as limiting the scope of the invention but as merely providing illustrations of some of the presently preferred embodiments of this invention. For example, the invention can incorporate an aphrodesiac such as yohimbe, a spermicidal such as nonoxynol 9, and a fungicidal such as clotrimazole. Thus the scope of the invention should be determined by the appended claims and their legal equivalents, rather than by the examples given.

What is claimed is:

1) compositions for female and male sexual arousal comprising:
a) a water miscible cream base,
b) an aphrodesiac mixed in said cream base.

2) Compositions as in claim 1 wherein said cream base comprises a mixture of approximately 80 parts water, 1 part cocoa butter, 2 parts Pro Lip HI 166.01, 2 parts glycerol monstearate, 2 parts acritamer carbomer, 1 part jojoba oil and 2 parts sodium behenoly lactylate.

3) Compositions as in claim 1 wherein said aphrodesiac is Yohimbe.

4) Compositions as in claim 1 wherein said aphrodesiac is Viagara TM.

5) Compositions as in claim 1 further comprising a fungicidal component.

6) Compositions as in claim 5 wherein said fungicidal component is Clotrimazole.

7) Compositions as in claim 1 further comprising a spermicidal component.

8) Compositions as in claim 7 wherein said spermicidal component is Nonoxynol 9.

9) A composition for male and female sexual arousal as in claim 1 comprising two thousand parts of said cream base and fifty parts Viagara TM.

10) A composition for male and female sexual arousal as in claim 1 comprising five thousand parts of said cream base and one hundred parts Viagara TM.

11) A composition for male and female sexual arousal as in claim 1 comprising two thousand parts of said cream base and approximately one hundred and forty parts yohimbe.

12) A composition for male and female sexual arousal as in claim 1 comprising five thousand parts of said cream base and approximately two hundred and eighty parts yohimbe.

13) A composition for male and female sexual arousal as in claim 12 for transdermal use further comprising one hundred parts of Nonoxynol 9, a spermicidal.

14) A composition for male and female sexual arousal as in claim 12 for transdermal use further comprising fifty parts Clotrimazole, a fungicidal.

15) A composition for male and female sexual arousal as in claim 10 comprising one hundred parts Nonoxynol 9, a spermicidal.

16) A composition for male and female sexual arousal as in claim 10 comprising fifty parts Clotrimazole, a fungicidal.

17) A composition for male and female sexual arousal as in claim 9 comprising one hundred parts Nonoxynol 9, a spermicidal.

18) A composition for lubricating a condom comprising two thousand parts of a water soluble cream base, fifty parts Viagara TM, and one hundred parts of Nonoxynol 9, a spermicidal.

19) A composition for lubricating a condom comprising two thousand parts of a water soluble cream base, two hundred parts of Yohimbe, and one hundred parts of Nonoxynol 9.

20) A method of use by a male of a cream based arousal composition wherein said male uses a condom lubricated with a minimum of about five milliliters of said cream based composition.

21) A method of use by a male or female of a cream based arousal composition as in claim 19 further comprising applying a layer of said arousal composition to genetalia of said male or female.
